(19) United States
(12) Patent Application Publication
(10) Pub. No.: US 2022/0259165 A1
(43) Pub. Date: Aug. 18, 2022

(54) S-OXIDES OF 2,3-DIARYL-2,3-DIHYDRO-4H-1,3-THIAZIN-4-ONES AND 2,3-DIARYL-1,3-THIAZEPAN-4-ONES AND METHODS FOR MAKING

(71) Applicant: The Penn State Research Foundation, University Park, PA (US)

(72) Inventor: Lee J. Silverberg, Allentown, PA (US)

(21) Appl. No.: 17/610,046

(22) PCT Filed: May 8, 2020
(86) PCT No.: PCT/US2020/032249

§ 371 Date: Nov. 9, 2021

Related U.S. Application Data
(60) Provisional application No. 62/846,046, filed on May 10, 2019.

(51) Int. Cl. C07D 281/02; C07D 279/08; C07D 279/06; C07D 513/04

(52) U.S. Cl. CPC C07D 281/02; C07D 513/04; C07D 279/06; C07D 279/08

(57) ABSTRACT

A compound with the following general formula and a general method of making this compound are provided: R¹, R², R³, R⁴, R⁵, R⁶, R⁷, R⁸, R⁹, R¹⁰, R¹¹, R¹², R¹³ and R¹⁴ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl.

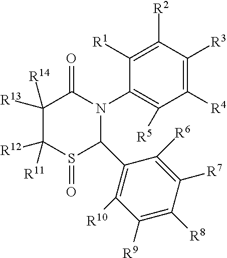

### CROSS-REFERENCE TO RELATED APPLICATIONS

This application claims the benefit of U.S. Provisional Patent Application No. 62/846,046, filed on May 10, 2019, which is incorporated by reference in its entirety.

### BACKGROUND OF THE INVENTION

### Field of the Invention

The present invention relates to S-oxides of a six-membered 1,3-thiazin-4-one system and S-oxides of a seven-membered 1,3-thiazapan-4-one ring system.

The 1,3-thiazin-4-ones are a group of six-membered heterocycles with a wide range of biological activity (Ryabukhin, Y. I., Korzhavina, O. B. & Suzdalev, K. F. Adv. Heterocycl. Chem. 1996, 66, 131-190). Surrey's research (Surrey, A. R., Webb, W. G.; Gesler, R. M. J. Am. Chem. Soc. 1958, 80, 3469-3471; Surrey, A. R. U.S. Pat. No. 3,082,209, 1963; Surrey, A. R. U.S. Pat. No. 3,093,639, 1963) resulted in the discovery of two drugs, the antianxiety and muscle relaxant chlorrnezanone [2-(4-chlorophenyl)-3-methyl-2,3,5,6-tetrahydro-4H-1,3-thiazin-4-one 1,1-dioxide] (O'Neil, M. J. Editor. The Merck Index, 14th ed., 2006, Whitehouse Station, N.J.: Merck & Co. Inc., p. 349; Tanaka, R. & Horayarna, N. X-Ray Struct. Anal. Online, 2005, 21, x57-x58) and muscle relaxant dichlormezanone [2-(3,4-dichlorophenyl)-3-methyl-2,3,5,6-tetrahydro-4H-1,3-thiazin-4-one 1,1-dioxide] (Elks, J. & Ganellin, C. R. Editors. Dictionary of Drugs, 1990, Cambridge, UK: Chapman and Hall, p. 382). These sulfones showed greater activity than the sulfides from which they were synthesized (Surrey, A. R., Webb, W. G.; Gesler, R. M. J. Am. Chem. Soc. 1958). Surrey also prepared a variety of other sulfoxides and sulfones of 3-alkyl-2-aryl-2,3,5,6-tetrahydro-4H-1,3-thiazin-4-ones (Surrey, A. R. U.S. Pat. No. 3,082,209, 1963; Surrey, A. R. U.S. Pat. No. 3,093,639, 1963). Surrey did not successfully synthesize any 2-aryl-3-aryl-2,3,5,6-tetrahydro-4H-1,3-thiazin-4-ones.

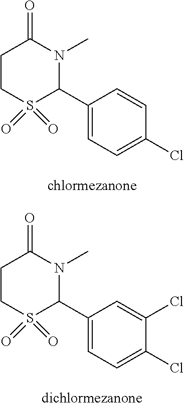 [SMILES: CN1C(=O)CCS(=O)(=O)C1c1ccc(Cl)cc1 ; CN1C(=O)CCS(=O)(=O)C1c1ccc(Cl)c(Cl)c1]

The seven-membered 1,3-thiazepan-4-one ring system is also of biological interest, as exemplified by the investigational compound omapatrilat (Graul, A., Leeson, P.; Castañer, J. Drugs Future, 1999, 24, 269-277; Robl, J. A., et al. J. Med. Chem. 1997, 40, 1570-1577; Tabrizchi, R. Curr. Opin. Investig. Drugs, 2001, 2, 1414-1422).

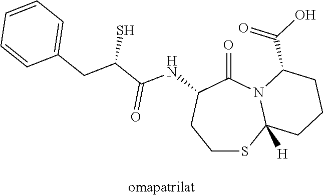 [SMILES: O=C(N[C@H]1CCS[C@H]2CCC[C@@H](C(=O)O)N2C1=O)[C@@H](S)Cc1ccccc1]

Sulfoxides and sulfones of 2,3-diaryl-2,3-dihydro-4H-1,3-thiazin-4-ones and 2,3-diaryl-1,3-thiazepan-4-ones, along with methods for creating these compounds, can lead to new therapeutics due to their biological activity and potential medicinal properties.

### SUMMARY OF THE INVENTION

One embodiment of this invention is directed to a compound of Formula I.

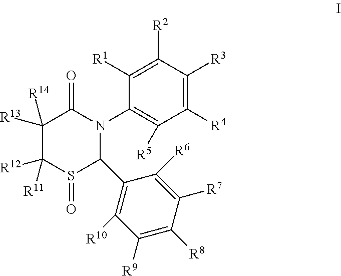

R¹, R², R³, R⁴, R⁵, R⁶, R⁷, R⁸, R⁹, R¹⁰, R¹¹, R¹², R¹³ and R¹⁴ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl. The pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups. Formula I is not

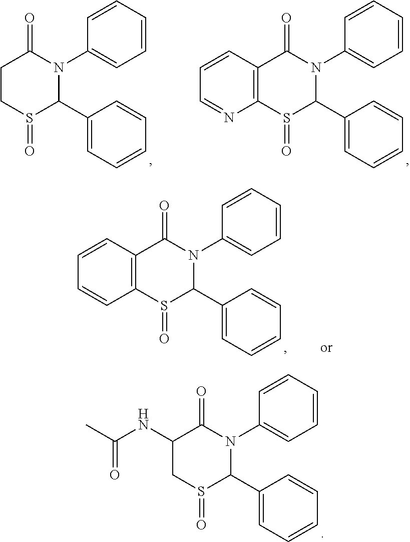

Another embodiment of this invention is directed to a compound of Formula II:

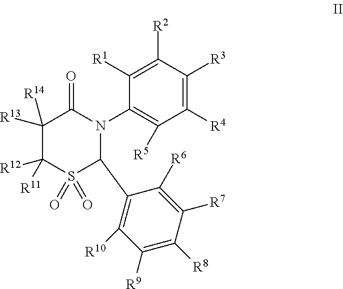

R¹, R², R³, R⁴, R⁵, R⁶, R⁷, R⁸, R⁹, R¹⁰, R¹¹, R¹², R¹³ and R¹⁴ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl. The pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups. And not all of R¹, R², R³, R⁴, R⁵, R⁶, R⁷, R⁸, R⁹, R¹⁰, R¹¹, R¹², R¹³ and R¹⁴ are hydrogen.

In another embodiment, the compound is

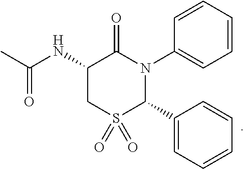 [SMILES: CC(=O)N[C@H]1CS(=O)(=O)[C@@H](c2ccccc2)N(c2ccccc2)C1=O]

In another embodiment, the compound is

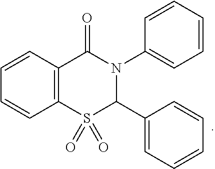 [SMILES: O=C1c2ccccc2S(=O)(=O)C(c2ccccc2)N1c1ccccc1]

In another embodiment, the compound is

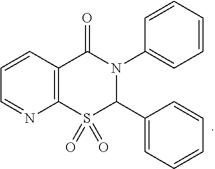 [SMILES: O=C1c2cccnc2S(=O)(=O)C(c2ccccc2)N1c1ccccc1]

Another embodiment of this invention is directed to a compound of Formula III:

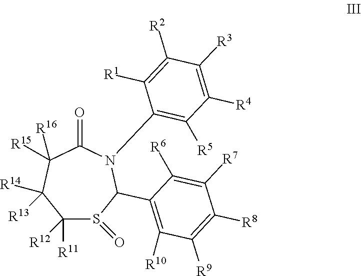

R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl. The pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups.

In another embodiment, the compound is

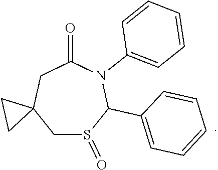 [SMILES: O=C1CC2(CC2)CS(=O)C(c2ccccc2)N1c1ccccc1]

Another embodiment of this invention is directed to a compound of Formula IV:

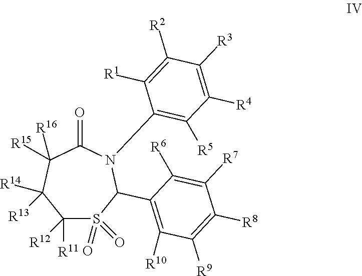

R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl. The pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups.

In another embodiment, the compound is

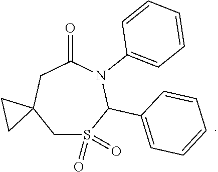 [SMILES: O=C1CC2(CC2)CS(=O)(=O)C(c2ccccc2)N1c1ccccc1]

Other aspects and advantages of the invention will be apparent from the following description and the appended claims.

### DETAILED DESCRIPTION OF THE INVENTION

### Definitions

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

The terms “a,” “an,” and “the” refer to “one or more” when used in this application, including the claims. The use of the word “a” or “an” when used in conjunction with the term “comprising” in the claims and/or the specification may mean “one,” but it is also consistent with the meaning of “one or more,” “at least one,” and “one or more than one.”

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and devices of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing physical dimensions, quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “alkyl” includes branched, straight chain and cyclic, substituted or unsubstituted saturated aliphatic hydrocarbon groups. Alkyl groups can comprise about 1 to about 24 carbon atoms (“C1-C24”), about 7 to about 24 carbon atoms (“C7-C24”), about 8 to about 24 carbon atoms (“C8-C24”), or about 9 to about 24 carbon atoms (“C9-C24”). Alkyl groups can also comprise about 1 to about 8 carbon atoms (“C1-C8”), about 1 to about 6 carbon atoms (“C1-C6”), or about 1 to about 3 carbon atoms (“C1-C3”). Examples of C1-C6 alkyl groups include, but are not limited to, methyl, ethyl, propyl, isopropyl, butyl, sec-butyl, tert-butyl, pentyl, isopentyl, neopentyl, hexyl, isohexyl, cyclohexyl, cyclohexylmethyl, cyclopropylmethyl and neohexyl radicals.

The term “aryl” includes a 6- to 14-membered monocyclic, bicyclic or tricyclic aromatic hydrocarbon ring system. Examples of an aryl group include phenyl and naphthyl.

The term “aralkyl” refers to an aryl-alkyl group wherein aryl and alkyl are as previously described.

The term “heteroaryl” includes an aromatic heterocycle ring of 5 to 14 members and having at least one heteroatom selected from nitrogen, oxygen and sulfur, and containing at least 1 carbon atom, including monocyclic, bicyclic, and tricyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

As used herein, the term “halogen” means F, Cl, Br or I.

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

The present invention is directed to classes of 2,3-diaryl-2,3-dihydro-4H-1,3-thiazin-4-ones I and II, and 2,3-diaryl-1,3-thiazepan-4-ones III and IV and methods to make them.

General Synthetic Procedure for Sulfoxides Formulas I and III:

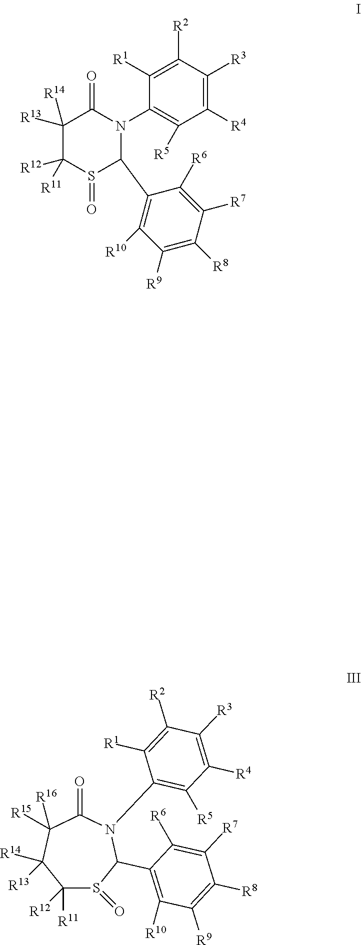

In compound I, R¹, R², R³, R⁴, R⁵, R⁶, R⁷, R⁸, R⁹, R¹⁰, R¹¹, R¹², R¹³ and R¹⁴ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl.

In compound III, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl.

The heterocycle (1.0 equiv.) was dissolved in methanol (8 mL/mmol). An aqueous solution of Oxone® (3.0 equiv. calculated as KHSO₅, 152.2 g mol⁻¹), in water (4 mL/3 mmol) was added dropwise at room temperature with vigorous stirring. After the addition, the reaction mixture was stirred and the reaction was followed by TLC. Water was added to the mixture to dissolve precipitates, and the mixture was extracted with 4 times with CH₂Cl₂ or ethyl acetate. The organic layers were combined and washed with water and then saturated NaCl. The solution was dried over sodium sulfate and concentrated under vacuum to give a crude solid. Further purification is described below.

### Example 1

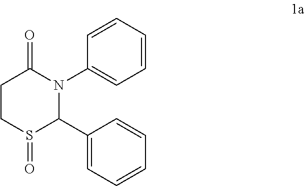 [SMILES: O=C1CCS(=O)C(c2ccccc2)N1c1ccccc1]

2,3-diphenyl-2,3,5,6-tetrahydro-4H-1,3-thiazin-4-one 1-oxide 1a: ¹H NMR of the crude product showed a diastereomeric ratio of 95:5. The product was purified by chromatography in a silica gel micro-column with mixtures of ethyl acetate and hexanes. 0.0251 g (45%). m.p.: 160-163° C. ¹H NMR (CDCl₃): δ (ppm): 7.51 (m, 2H), 7.46 (m, 3H), 7.38 (m, 2H), 7.32 (m, 3H), 5.98 (d, 1H, J=2.1 Hz), 3.36 (m, 1H), 2.99 (m, 1H), 2.85 (m, 2H). Structure confirmed by X-Ray crystallography.

### Example 2

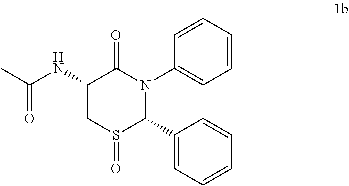 [SMILES: CC(=O)N[C@H]1CS(=O)[C@@H](c2ccccc2)N(c2ccccc2)C1=O]

N-[(1S,2S,5R)-1,4-dioxo-2,3-diphenyl-1λ⁴,3-thiazinan-5-yl]acetamide 1b: ¹H NMR of the crude product showed a diastereomeric ratio of 37:63. The product was purified by chromatography in a silica gel micro-column with mixtures of ethyl acetate and hexanes. 0.0558 g (96%). m.p. 176-179° C. Structure confirmed by X-Ray crystallography.

### Example 3

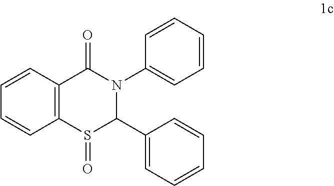 [SMILES: O=C1c2ccccc2S(=O)C(c2ccccc2)N1c1ccccc1]

2,3-diphenyl-2,3-dihydro-4H-1,3-benzothiazin-4-one 1-oxide 1c: ¹H NMR of the crude product showed a diastereomeric ratio of 93:7. Recrystallized from CH₂Cl₂/hexanes. 0.0429 g (55%). m.p. 208-209° C. H NMR (CDCl₃): δ (ppm): 7.72 (m, 1H), 7.55, m, 2H), 7.46 (m, 5H), 7.36 (m, 1H), 7.29 (m, 6H), 6.28 (s, 1H). Structure confirmed by X-Ray crystallography.

### Example 4

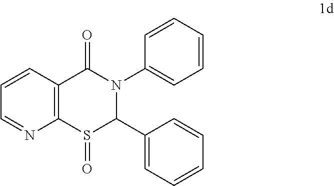 [SMILES: O=C1c2cccnc2S(=O)C(c2ccccc2)N1c1ccccc1]

2,3-diphenyl-2,3-dihydro-4H-pyrido[3,2-e]-[1,3]thiazin-4-one 1-oxide 1d: ¹H NMR of the crude product showed a diastereomeric ratio of 84:16. The product was purified by chromatography in a silica gel micro-column with mixtures of ethyl acetate and hexanes. 0.0226 g (43%). m.p. 177-179° C. ¹H NMR (CDCl₃): δ (ppm): 8.70 (m, 2H), 7.63 (s, 1H), 7.48 (s, 4H), 7.36 (m, 6H), 6.34 (s, 1H). Structure confirmed by X-Ray crystallography.

### Example 5

General Synthetic Procedure for Sulfones Formulas II and IV:

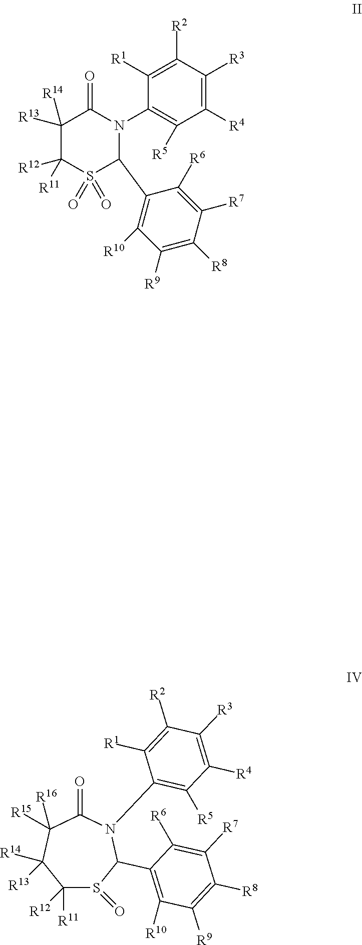

In compound II, R¹, R², R³, R⁴, R⁵, R⁶, R⁷, R⁸, R⁹, R¹⁰, R¹¹, R¹², R¹³ and R¹⁴ are each independently selected from the group that includes hydrogen, halogen, nitro, cyano, amido, pyridyl, alkyl, aryl, alkoxy, cycloalkyl, heteroalkyl, heterocyclyl, aralkyl, heteroaryl and heteroaralkyl.

In compound IV, R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that includes H, halogen, nitro, cyano, amido, pyridyl, alkyl, aryl, alkoxy, cycloalkyl, heteroalkyl, heterocyclyl, aralkyl, heteroaryl and heteroaralkyl.

The heterocycle (0.267 mmol) was dissolved in glacial acetic acid (1.2 mL). An aqueous solution of KMnO₄ (0.535 mmol in 1.45 mL water was added dropwise at room temperature with vigorous stirring. The reaction was followed by TLC. Solid sodium bisulfite (NaHSO₃/Na₂S₂O₅) was added until the solution remained colorless. 1.45 mL of water was added and stirred for 10 min. The mixture was extracted with CH₂Cl₂ (3×5 mL). The organics were combined and washed once with sat. NaCl. The solution was dried over Na₂SO₄ and filtered. The product was purified by chromatography in a silica gel micro-column.

### Example 6

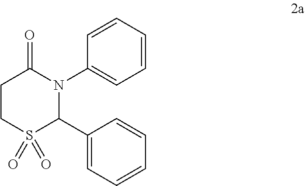 [SMILES: O=C1CCS(=O)(=O)C(c2ccccc2)N1c1ccccc1]

2,3-diphenyl-2,3,5,6-tetrahydro-4H-1,3-thiazin-4-one 1,1-dioxide 2a: 0.0534 g (70%). m.p. 145-148° C. ¹H NMR (CDCl₃): δ (ppm): 7.58 (m, 2H), 7.50 (m, 3H), 7.36 (m, 3H), 7.30 (m, 1H), 7.23 (m, 2H), 5.61 (s, 1H), 3.48 (m, 1H), 3.99 (m, 3H).

### Example 7

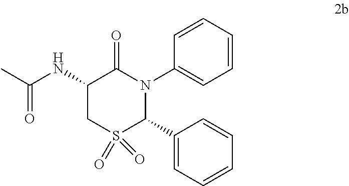 [SMILES: CC(=O)N[C@H]1CS(=O)(=O)[C@@H](c2ccccc2)N(c2ccccc2)C1=O]

N-[(2S,5R)-1,1,4-trioxo-2,3-diphenyl-1λ⁶,3-thiazinan-5-yl]acetamide 2b: 0.0762 g (80%). m.p. 170-194° C. (decomposition). ¹H NMR (CDCl₃): δ (ppm): 7.37 (m, 2H), 7.30 (m, 1H), 7.24 (m, 2H), 7.17 (m, 3H), 6.95 (m, 2H), 5.81 (s, 1H), 5.12 (dt, 1H, J=12, 6.1 Hz), 3.92 (m, 1H), 3.54 (m, 1H), 2.03 (d, 1H, J=1.8 Hz). Structure confirmed by X-Ray crystallography.

### Example 8

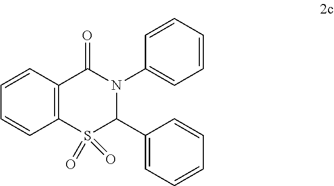 [SMILES: O=C1c2ccccc2S(=O)(=O)C(c2ccccc2)N1c1ccccc1]

2,3-diphenyl-2,3-dihydro-4H-1,3-benzothiazin-4-one 1,1-dioxide 2c: 0.050 g (54%). m.p. 163-165° C. ¹H NMR (CDCl₃): δ (ppm): 8.29 (d, 1H, J=7.7 Hz), 7.70 (m, 2H), 7.60 (m, 1H), 7.32 (m, 2H), 7.25 (m, 8H), 5.79 (m, 1H).

### Example 9

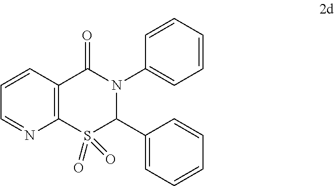 [SMILES: O=C1c2cccnc2S(=O)(=O)C(c2ccccc2)N1c1ccccc1]

2,3-diphenyl-2,3-dihydro-4H-pyrido[3,2-e]-[1,3]thiazin-4-one 1-oxide 2d: 0.0691 g (74%). m.p.: 210-211° C. (decomposition). ¹H NMR (CDCl₃): δ (ppm): 8.77 (d, J=4.7 Hz, 1H), 8.62 (d, J=7.9 Hz, 1H), 7.67 (dd, J=8.0, 4.7 Hz, 1H), 7.31 (m, 9H), 5.88 (s, 1H).

### Example 10

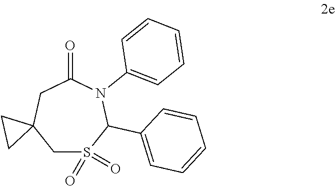 [SMILES: O=C1CC2(CC2)CS(=O)(=O)C(c2ccccc2)N1c1ccccc1]

6,7-diphenyl-5?⁶-thia-7-azaspiro[2.6]nonane-5,5,8-trione 2e: 70% yield. m.p. 186.6-187.7° C. (decomposition). ¹H NMR (CDCl₃): δ (ppm): 7.72 (s, 3H), 7.44 (m, 3H), 7.35 (m, 4H), 7.29 (m, 1H), 6.05 (s, 1H), 3.50 (d, 1H, J=14.6 Hz), 2.93 (d, 1H, J=14.6 Hz), 2.86 (m, 1H), 2.56 (bs, 1H), 1.02 (m, 2H), 0.77 (t, 2H, J=7.8 Hz). Structure confirmed by X-Ray crystallography.

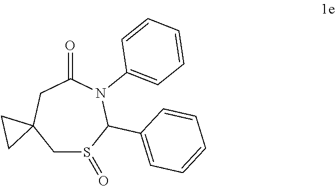 [SMILES: O=C1CC2(CC2)CS(=O)C(c2ccccc2)N1c1ccccc1]

6,7-diphenyl-5λ⁴-thia-7-azaspiro[2.6]nonane-5,8-dione 1e: In one experiment, this was isolated along with 2e from the KMnO₄ reaction. m.p. 193-194° C. ¹H NMR (CDCl₃): δ (ppm): 7.40-7.10 (m, 7H), 7.27 (m, 2H), 7.19 (d, 1H, J=7.0 Hz), 6.33 (s, 1H), 3.18 (d, 1H, J=14.0 Hz), 3.04 (d, 1H, J=13.4 Hz), 2.74 (bs, 2H), 1.08-0.98 (m, 2H), 0.71 (m, 1H), 0.60 (m, 1H).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

All references cited herein including those below are hereby incorporated by reference in their entirety.
H. P. Yennawar, Z. Yang, and L. J. Silverberg, “Crystal structure of rac-2,3-diphenyl-2,3,5,6-tetrahydro-4H-1,3-thiazin-4-one 1-oxide,” Acta Cryst., Sect. E: Crystallogr. Commun. 2016, E72, 1541-1543.
H. P. Yennawar, D. J. Noble, and L. J. Silverberg, “Crystal structure of (1S,2S,5R)-5-(acetylamino)-4-oxo-2,3-diphenyl-1,3-thiazinan-1-ium-1-olate,” Acta Cryst., Sect. E: Crystallogr. Commun. 2017, E73, 1417-1420.
H. P. Yennawar, D. J. Noble, Z. Yang, and L. J. Silverberg, “rac-2,3-Diphenyl-2,3-dihydro-4H-pyrido[3,2-e][1,3]thiazin-4-one 1-oxide,” IUCrData 2017, 2, x171112.
H. P. Yennawar, R. F. Fox, Q. J. Moyer, Z. Yang, and L. J. Silverberg, “Crystal structure of 2,3-diphenyl-2,3-dihydro-4H-1,3-benzothiazin-4-one 1-oxide,” Acta Cryst., Sect. E: Crystallogr. Commun. 2017, E73, 1189-1191.

We claim:

1. A compound of Formula I: 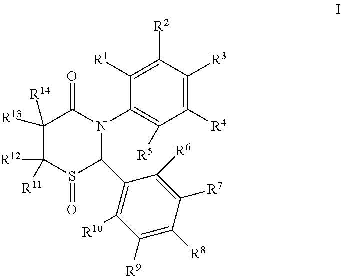
wherein R¹, R², R³, R⁴, R⁵, R⁶, R⁷, R⁸, R⁹, R¹⁰, R¹¹, R¹², R¹³ and R¹⁴ are each independently selected from the group consisting of hydrogen, halogen, nitro, cyano, amido, pyridyl, alkyl, aryl, alkoxy, cycloalkyl, heteroalkyl, heterocyclyl, aralkyl, heteroaryl and heteroaralkyl, wherein the pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups, and
wherein Formula I is not

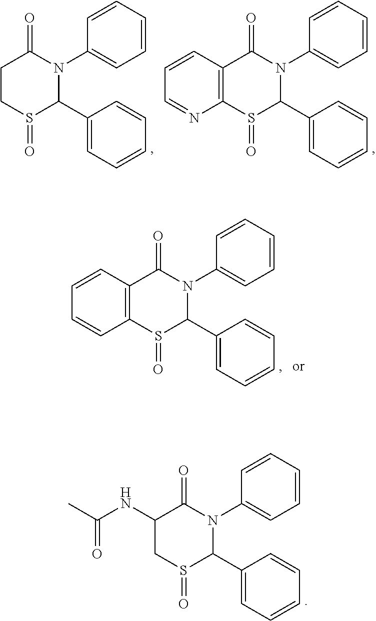

2. A compound of Formula II: 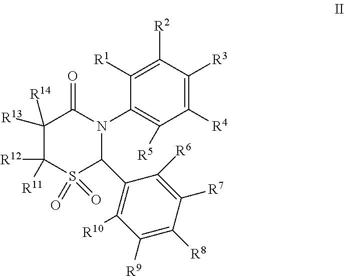
wherein R¹, R², R³, R⁴, R⁵, R⁶, R⁷, R⁸, R⁹, R¹⁰, R¹¹, R¹², R¹³ and R¹⁴ are each independently selected from the group consisting of hydrogen, halogen, nitro, cyano, amido, pyridyl, alkyl, aryl, alkoxy, cycloalkyl, heteroalkyl, heterocyclyl, aralkyl, heteroaryl and heteroaralkyl,
wherein the pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups, and
wherein not all of R¹, R², R³, R⁴, R⁵, R⁶, R⁷, R⁸, R⁹, R¹⁰, R¹¹, R¹², R¹³ and R¹⁴ are hydrogen.

3. The compound of claim 2, wherein the compound is

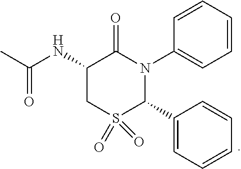 [SMILES: CC(=O)N[C@H]1CS(=O)(=O)[C@@H](c2ccccc2)N(c2ccccc2)C1=O]

4. The compound of claim 2, wherein the compound is

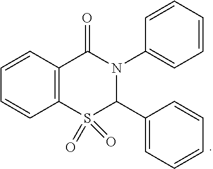 [SMILES: O=C1c2ccccc2S(=O)(=O)C(c2ccccc2)N1c1ccccc1]

5. The compound of claim 2, wherein the compound is

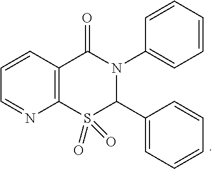 [SMILES: O=C1c2cccnc2S(=O)(=O)C(c2ccccc2)N1c1ccccc1]

6. A compound of Formula III: 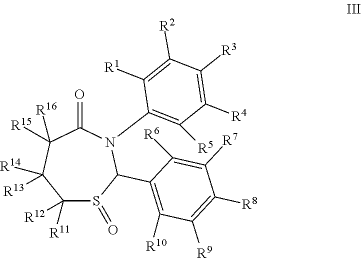
wherein R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that consists of hydrogen, halogen, nitro, cyano, amido, pyridyl, alkyl, aryl, alkoxy, cycloalkyl, heteroalkyl, heterocyclyl, aralkyl, heteroaryl and heteroaralkyl, and
wherein the pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups.

7. The compound of claim 6, wherein the compound is

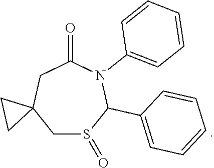 [SMILES: O=C1CC2(CC2)CS(=O)C(c2ccccc2)N1c1ccccc1]

8. A compound of Formula IV: 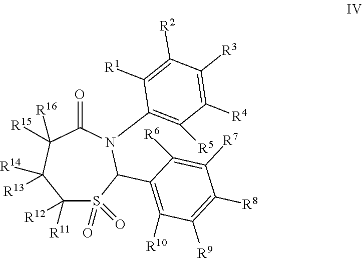
wherein R¹, R², R³, R⁴, R⁵, R⁶, R⁷, R⁸, R⁹, R¹⁰, R¹¹, R¹², R¹³, R¹⁴, R¹⁵ and R¹⁶ are each independently selected from the group that consists of hydrogen, halogen, nitro, cyano, amido, pyridyl, alkyl, aryl, alkoxy, cycloalkyl, heteroalkyl, heterocyclyl, aralkyl, heteroaryl and heteroaralkyl, and
wherein the pyridyl, alkyl, aryl, alkoxy, cycloalkyl, heteroalkyl, heterocyclyl, aralkyl, heteroaryl and heteroaralkyl may be optionally substituted with one or more of methyl, ethyl, halogen, nitro, methoxy, or cyano groups.

9. The compound of claim 8, wherein the compound is 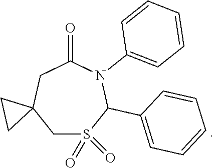 [SMILES: O=C1CC2(CC2)CS(=O)(=O)C(c2ccccc2)N1c1ccccc1]
